(19) United States
(12) Patent Application Publication
(10) Pub. No.: US 2008/0057553 A1
(43) Pub. Date: Mar. 6, 2008

(54) FERMENTATION PROCESSES AND SYSTEMS

(76) Inventor: Robert E. Cadwalader, Glen Ellyn, IL (US)

Correspondence Address: MICHAEL P. MAZZA, LLC, 686 CRESCENT BLVD., GLEN ELYN, IL, 60137, US

(21) Appl. No.: 11/468,914

(22) Filed: Aug. 31, 2006

(51) Int. Cl. C12P 7/20; C12P 7/16; C12P 7/06; C12M 3/00

(52) U.S. Cl. 435/159; 435/289.1; 435/160; 435/161

(57) ABSTRACT

A system and method for fermenting substrates using a continuous processing system, including agricultural and industrial by-products such as dairy products, cellulosic-based products, and sugar-based products, into useful target products such as ethanol. One preferred system includes at least two reaction vessels and one or more membrane filters. The reaction vessels may each contain a mash with substrate and fermentation microorganisms useful in converting the substrate to the target product. The membrane filter(s) may be located downstream from reaction vessels and used to separate the mash into a retentate and a permeate. The microorganisms may be generally retained in the retentate and returned to their originating reaction vessel, while the permeate, not including the microorganisms, may be introduced to the next reaction vessel in series.

### FIELD OF THE INVENTION

The present invention relates generally to fermentation processes and systems for utilizing such processes. More specifically, the invention relates to fermentation processes and systems capable of processing substrates into useful target products.

### BACKGROUND OF THE INVENTION

Industrial ethanol production is generally based on either ethylene conversion of fossil fuels such as oil and coal, or fermentation of carbohydrate-containing materials, such as agricultural products. Industrial fermentation of agricultural products for the production of target products, such as ethanol, is generally accomplished through batch processing.

Various economic and environmental factors have increased the demand for ethanol, and have created a complementary desire to reduce the use of fossil fuels. Environmental and economic factors drive a desire to decrease the quantity of discarded agricultural byproducts. The food industry disposes of significant quantities of fermentable material every year for lack of an efficient low cost means of fermenting its affluent. In particular, small-scale producers of fermentable byproducts lack an efficient means for converting the byproducts into useful target products, such as ethanol.

Some known systems and methods disclose the continuous processing of agricultural products, including: “Hollow fibre bioreactor for ethanol production: application to the conversion of lactose by Kluyveromyces fragilis,” by Mehaia, et al., Enzyme Microb. Technol., 1984, Vol. 6, March, p. 117-120; “A high-performance membrane bioreactor for continuous fermentation of lactose to ethanol,” by Cheryan, et al., Biotechnology Letters, Vol. 5, No. 8, p. 519-524 (1983); “Ethanol from whey: continuous fermentation with a catabolite repression-resistant saccharomyces cerevisiae mutant,” by Terrell, et al, Applied and Environmental Microbiology, September 1984, p. 577-580; U.S. Pat. No. 4,460,687 issued to Ehnstrom; and U.S. Pat. No. 5,688,674 issued to Choi, et al. Each of these publications is entirely incorporated herein by reference.

Although continuous processing promises many benefits, such as increased and less expensive production, the industry has encountered problems with regard to continuous processing related to limitations upon product yield, yeast maintenance as the percentage of target product increases in the processing vessels, and contamination and cross-contamination of microorganisms between reaction vessels. In addition, the industry has encountered problems associated with the cost and inflexibility associated with the use of large bioreactor vessels.

### DEFINITION OF CLAIM TERMS

The following terms are used in the claims of the patent as filed and are intended to have their broadest meaning consistent with the requirements of law. Where alternative meanings are possible, the broadest meaning is intended. All words used in the claims are intended to be used in the normal, customary usage of grammar and the English language.

“Contaminating microorganisms” means reactive microorganisms that do not participate in a useful manner, or that participate in a harmful manner, with the production of a target product from a substrate.

“Continuous flow” means a fermentation process in which target product is output from the system while most of the mash remains in one or more reaction vessels, and in which emptying of reaction vessels is generally not required to maintain production of the target product. “Continuous flow” includes fermentation systems in which the fermentation microorganism cell mass in a reaction vessel is maintained at a viable level while the target product is removed from the system.

“Fermentation microorganisms” means reactive microorganisms involved in a microbial-controlled production of a target product from an organic or inorganic substrate.

“Fluid” refers to both liquid and gaseous phases.

“Mash” means the contents of a reaction vessel, which may include: feed substrate, nutrients, fermentation microorganisms, water, minerals, the target product, and miscellaneous metabolic by-products in small quantities.

“Permeate” means materials flowing through a first bioreactor vessel, and passing through a membrane filter for entry to a second bioreactor vessel downstream of the first bioreactor vessel.

“Reactive microorganisms” means microorganisms that react with a substrate, including both fermentation microorganisms and contaminating microorganisms.

“Retentate” means materials flowing through a bioreactor vessel that include substances retained by a membrane filter, and that are returned to that bioreactor vessel.

“Sterilized” and “sterilization” means a microbial contamination capability sufficiently low so as to cause no meaningful harm to the fermentation process or to the microorganisms selected to carry out the fermentation process.

“Substrate” means a material capable of being at least partially converted into a target product by fermentation microorganisms.

### SUMMARY OF THE INVENTION

The present invention provides processes and apparatus for continuous processing of food agricultural substances such as dairy products and non-food substances such as cellulosic-based materials into useful yields, such as ethanol. In a preferred method according to the present invention,

In one preferred apparatus, the invention may include a plurality of reaction vessels and a membrane filter. A first reaction vessel may hold a first mash that includes a substrate and fermentation microorganisms for converting the substrate to a target product. The membrane filter may be located downstream from the first reaction vessel and may separate the first mash into a first retentate and a first permeate. The microorganisms may be generally retained in the retentate and returned to the first reaction vessel while the first permeate may be introduced to the second reaction vessel. In the preferred embodiment, each separate reaction vessel may retain the microorganisms initially resident in that separate vessel.

The invention addresses the problems encountered in the industry related to the inherent limitations on product yield, yeast maintenance, and contamination. The invention also reduces the amount of water required for processing as compared to prior art batch processing systems, reduces the size of reaction vessels required, and allows for lower operating costs through greater automation of process controls.

In a preferred embodiment, a continuous-flow fermentation system for providing a target product from a substrate is disclosed. The system includes at least first and second reaction vessels in selective fluid communication. The first reaction vessel may be charged with a first mash that includes the substrate and reactive microorganisms. The reactive microorganisms may include fermentation microorganisms useful for converting the substrate into the target product. The second reaction vessel may be charged with a second mash that also includes a substrate and reactive microorganisms. The second reaction vessel may receive a selective portion of the first mash from the first reaction vessel. A membrane filter may be located between the first and second reaction vessels, and may be configured to separate the first mash from the first reaction vessel into at least a first retentate and a first permeate. A microorganism refiner, such as a membrane filter or a centrifugal separator, may be located downstream from the second reaction vessel. The reactive microorganisms within the first reaction vessel may be generally retained in the first retentate by the membrane filter and returned to the first reaction vessel. The first permeate, which is substantially free of microorganisms, may be introduced to the second reaction vessel. The reactive microorganisms within the second reaction vessel may form a second retentate, which may be generally retained in the second reaction vessel by the microorganism refiner. Each separate reaction vessel may be capable of substantially retaining the reactive microorganisms initially resident in that separate vessel.

In one embodiment, a portion of either the first and/or the second retentate may be removed from its corresponding reaction vessel, the reactive microorganisms may be removed from it, and the remainder of the retentate may be transferred back to one of the reaction vessels.

The reactive microorganisms in the reaction vessels may be of the same type of microorganism or of different types. Different types may be used, for example, because one type of microorganism is capable of more efficiently processing the substrate into the target product in the presence of relatively greater concentrations of target product than the second type of fermentation microorganism. Other reasons may drive the selection of different types of microorganisms. Also, each reaction vessel may house different microorganisms, or combinations of different microorganisms may be contained within the same reaction vessel.

More than two reaction vessels may be used. Additional reaction vessels may be placed in series or in parallel with the first two reaction vessels. Microorganism refiners such as membrane filters and/or centrifugal separators may be located downstream of each of the reaction vessels, so that any contaminating microorganisms are contained within the vessel and not allowed to spread to other vessels.

Sterilized oxygen may be used to grow the fermentation microorganism. A nitrogen-containing substance such as ammonia or ammonium salts may be used to control the pH of the mash in each bioreactor.

As non-limiting examples, the substrate may include one or more of the following: lactose; whey; whey permeate; corn; wheat; rye; rice; potatoes; artichokes; sugar beets; sugarcane; fruits; plant fiber; wood by-products; paper; and/or grasses. Also as non-limiting examples, the target product may include, but is not limited to, one or more of the following: ethanol; propanol; isopropanol; butanol; and/or acetone. The target product may be produced in an aerobic or an anaerobic reaction. The membrane filter may include, but is not limited to, one or more of the following materials or classes of materials or substances: cellulosic; polyvinylidene fluoride; polyether sulfone; polysulfone; ceramic; sintered stainless steel; and graphite.

The membrane filter may be configured in one or more of the following shapes: hollow fiber; flat sheet spiral; flat sheet plate; frame and/or tubular. The membrane filter characteristics may be altered during the operation of the system by an electrical charge directed on or through the membrane, for example. The membrane filter may have pores that have a diameter between 150 angstroms and 1 micron. The membrane filter may have an isotropic or anisotropic morphology. The membrane filter may be configured to operate at pressures between 5 to 150 psig.

A method of continuously fermenting a mash including a substrate and reactive microorganisms to convert the substrate into a useful target product also forms a part of the present invention. In a preferred method, at least first and second reaction vessels are provided in selective fluid communication with each other in a continuous flow system. The reaction vessels may each contain a mash with a substrate and reactive microorganisms useful in converting the substrate into a target product. A membrane filter may be located downstream from the first reaction vessel and configured to receive at least a portion of the mash and to separate the mash into at least a first retentate and a first permeate. The reactive microorganisms within the first reaction vessel may be generally retained in the retentate by the membrane filter and returned to the first reaction vessel. A microorganism refiner may be located downstream from the second reaction vessel and configured to receive at least a portion of its mash and to separate this mash into a second retentate and a second permeate. The reactive microorganisms within the second reaction vessel may be generally retained in the second retentate by the microorganism refiner and returned to the second reaction vessel. The first permeate, which is substantially free of microorganisms, may be introduced to the second reaction vessel. In this way, each separate reaction vessel may substantially retain the microorganisms initially resident in that separate vessel.

Target product may be recovered from vapors emanating from the reaction vessels. This recovery step may include steps of directing the vapors through a condenser, and then either directing the condensed vapor into the last reactor vessel, or directing the condensed target product through a target product separation processor. The vapors remaining after condensation may pass through a sterilizing vent filter.

Other systems, methods, features, and advantages of the present invention will be, or will become, apparent to one having ordinary skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The novel features which are characteristic of the invention are set forth in the appended claims. The invention itself, however, together with further objects and attendant advantages thereof, can be better understood by reference to the following description taken in connection with the accompanying drawings, in which:

FIG. 1 is a schematic diagram of a bioreactor system in which fermentation microorganisms may be recovered and returned to a number of bioreactors throughout the system;

FIG. 2 is an illustrative diagram of a filtering membrane system that may be employed in the bioreactor system of FIG. 1;

FIG. 3 is a schematic diagram of a bioreactor system in which the fermentation microorganisms may be recovered and returned to the first bioreactor in a series of bioreactors; and

FIG. 4 is a flow chart illustrating a method of continuous processing of substrates, such as dairy products, into useful yields, such as ethanol.

The components in the drawings are not necessarily to scale, emphasis instead being placed upon clearly illustrating the principles of the present invention. In the drawings, like reference numerals designate corresponding parts throughout the several views.

### DETAILED DESCRIPTION OF PREFERRED EMBODIMENTS

Set forth below is a description of what are believed to be the preferred embodiments and/or best examples of the invention claimed. Future and present alternatives and modifications to the preferred embodiments are contemplated. Any alternatives or modifications which make insubstantial changes in function, in purpose, in structure, or in result are intended to be covered by the claims of this patent.

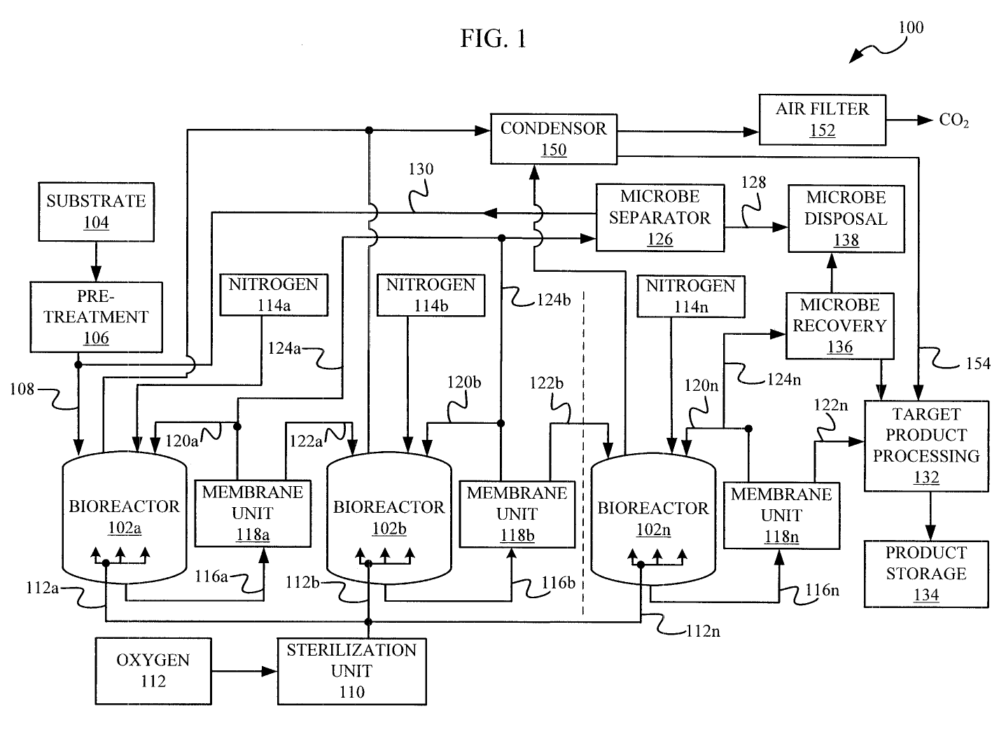

Referring first to FIG. 1, a bioreactor system 100 is shown in which incoming substrate may be at least partially processed into a target product and fermentation microorganisms may be recovered and returned to a number of bioreactors throughout the system 100. For example, fermentation microorganisms may be returned to the three reactive vessels, bioreactors 102a, 102b, and 102n shown in FIG. 1. Though three bioreactors are illustrated in FIG. 1, the actual number of bioreactors that may be incorporated into system 100 is unlimited and bioreactors may be added in series, and in parallel, to accommodate individual needs and production goals.

Bioreactor vessel sizes of 200 gallons to 50,000 gallons are envisioned for advantageous use, for example, although smaller or larger vessel sizes may be used. While it is envisioned that processing stoppage and cleaning between continuous processing runs will be required, it is currently envisioned that continuous processing runs of two weeks or more may be permissible.

In a fermentation process, exclusion of competing microorganisms is generally important. Among the sources of potential contamination, fermentation systems are vulnerable to contamination from incoming substrate, and from cross-fermentation between bioreactor vessels. In the case of lactose fermentation to produce a target product of ethanol, it may be important to inhibit the growth of microorganisms that would compete with fermentation yeasts, bacteria, and other microorganisms such as, but not limited to: Saccharomyces fragilis, Saccharomyces cerevisiae, Kluyveromyces fragilis, Kluyveromyces marxianus, Candida pseudotropicalis, Candida kefyr, Candida blankii, Candida valida, Torulopsis cremoris, Zymomonas mobilis, and genetic modifications of such organisms. For a discussion of efforts to process lactose into ethanol see “Hydrolysis of lactose in whey permeate for subsequent fermentation to ethanol,” by Cote, et al., J. Dairy Sci. Vol. 87, 1608-1620, which is entirely incorporated herein by reference.

In order to address potential contamination from incoming substrate, the bioreactor system 100 includes a pre-treatment unit 106 that receives substrate feed from the substrate feeder 104. The substrate feeder 104 may be a portable unit that can be loader at an agricultural production site and transported to the location of the other components of system 100.

In system 100, the substrate may be a carbohydrate agricultural product, or byproduct, which may be placed in the substrate feeder 104 prior to pre-treatment. In general where the target product is ethanol, the higher the carbohydrate percentage of the substrate, the greater the potential production of ethanol where other factors are constant. Substrate feed for use with the process invention may include a number of starch-, sugar- and cellulosic-based products and by-products. As non-limiting examples, starch-based substrates which may be used include, but are not limited to: lactose, whey, whey permeate, corn, wheat, rye, rice, potatoes, and artichokes. Sugar-based substrates which may be used include, but are not limited to: sugar beets, sugarcane, and fruits. Cellulosic-based substrates which may be used include, but are not limited to: wood by-products, wood fiber, plant fiber, paper and various grasses (e.g., prairie grass), as some examples.

Some different considerations come into play if different substrates are used, other than lactose. For example, if cellulosic-based substrates are used, a two-step process is necessary, as the cellulosic material will first be converted into sugar, and the sugar will then be fermented into alcohol. These two steps may occur in the same reactor vessel, or may be caused to occur in consecutive, different vessels, for example.

In one preferred embodiment, the pre-treatment unit 106 may receive substrate feed at solid concentration levels such as 4-25% solids by weight. In the pre-treatment unit 106 the substrate may be sterilized through a number of processes such as, but not limited to, thermal pasteurization, thermal sterilization, and sterilization filtration. In some applications, sterilization filtration may be accomplished by using membranes having pore sizes of 0.2 microns or less. Sterilization as used here does not necessarily mean the complete elimination of microbial contaminants, but also references a microbial contamination capability sufficiently low so as to not cause meaningful have to the fermentation process or to the microorganisms selected to carry out the fermentation process.

Chemical stabilization of the fermentation process may be enhanced by introducing sterilizing chemicals to the substrate to exclude non-yeast organisms and other organisms that may compete with the desired fermentation microbe. Sterilizing chemicals include, but are not limited to, hydrogen peroxide and sodium sulfite. In the case where hydrogen peroxide and sodium sulfite have been employed for chemical stabilization, effective concentrations have been found between 10 and 500 parts per million. When hydrogen peroxide is used, a catalyst may also be employed to break the hydrogen peroxide down into water and oxygen. The stabilizing chemicals may be introduced prior to the first reactor and/or into any or all bioreactors individually or collectively prior to, or during, the fermentation process.

First bioreactor 102a may receive sterilized substrate, sterilized oxygen, sterilized nutrients, fermentation microorganisms, and substrate return. Processing line 108 may be used to conduct sterilized substrate to first bioreactor 102a. A fermentation microorganism, such as yeast, may be input to bioreactor 102a before, during or after sterilized substrate is introduced to bioreactor 102a.

In some embodiments, fermentation microorganisms may be added when the bioreactor vessel is 10% full with substrate. Oxygen 112, or gas or fluid containing oxygen, may be introduced through oxygen sterilization unit 110. Oxygen sterilization unit 110 provides sterilized oxygen, with or without other accompanying sterilized gases or liquids, to bioreactor 102a through processing line 112a.

In one preferred embodiment, after loading the bioreactors 102a, 102b, 102n and during operation of the system 100, a portion of the content of the bioreactor, the “mash,” is continuously removed from the bioreactor, processed, and portions are returned to the bioreactor. In other embodiments, the mash may be periodically and/or intermittently removed from the bioreactor, processed, and returned to the bioreactor. In some embodiments the process of removing a portion of the mash may commence after the bioreactor is 90% full with substrate.

A portion of the contents of bioreactor 102a, the first mash, is conducted from bioreactor 102a, travels through processing line 116a, and is introduced to a membrane unit 118a. Membrane unit 118a separates the first mash into a first retentate and a first permeate. First retentate is returned to bioreactor 102a through processing line 120a. First permeate is introduced to bioreactor 102b through processing line 122a.

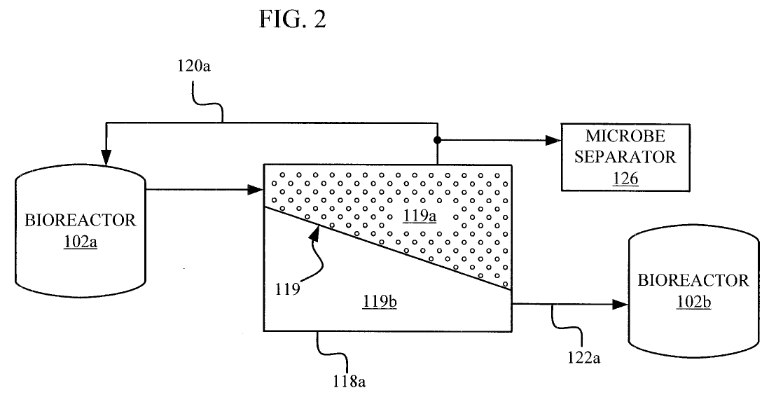

FIG. 2 is an illustrative diagram of the membrane unit 118a of FIG. 1. Membrane unit 118a includes a membrane 119 having a porous surface illustrated by pores 119a. Membrane unit 118a may include a variety of membrane filters including, but not limited to: organic, inorganic, isotropic, anisotropic and cross-flow/tangential flow membranes. If organic, membrane 119 may be composed of materials such as, but not limited to: cellulosic materials, polyvinylidene fluoride, polyether sulfone, and polysulfone. If inorganic, the membrane 119 may be composed of materials such as, but not limited to: ceramic, sintered stainless steel, and graphite. Although illustrated as having a straight diagonal surface in FIG. 2, membrane 119 may be configured in many different shapes, such as but not limited to hollow fiber, flat sheet spiral, flat sheet plate and frame, and tubular. In some embodiments, membrane characteristics may be altered during the operation of the system 100, such as by using an electrical charge to alter membrane characteristics. (Depending upon the membrane and charging process employed, the membrane may be charged directly, for example, or a charge may be placed in solution and migrated to the membrane, etc.) It may be preferred to employ more than one type of membrane configuration in combination to provide the desired separations for the process.

Pores 119a may be of a shape and/or size configured to limit the passage of microorganisms from one portion of the membrane unit 118a to another, and thus from one bioreactor to another. For example, pores 119a may be circular and have a diameter between 150 angstroms and 1 micron. Operating pressures for the membrane units may vary, and typically may vary from 5 to 150 psig. Some features of porous membranes for separating organisms are described in U.S. Pat. No. 3,580,840 issued to Uridil which is fully incorporated herein by reference.

In operation, membrane 119 provides a barrier through which microorganisms are substantially restricted from passing from one portion of the membrane unit 118a to another portion of the membrane unit 118a. Membrane unit 118a allows microorganism concentration to be increased and maintained at optimal levels such as, for example, 0.2 to 150 grams dry weight per liter, in a bioreactor vessel. For example, in one embodiment membrane 119 may limit passage of microorganisms in order to maintain 0.2. to 120 grams per liter of fermentation microorganisms in bioreactor 102a. In other embodiments, membrane 119 may limit passage of microorganisms in order to maintain greater or lesser grams per liter of fermentation microorganisms in bioreactor 102a. This enables microorganism concentrations to be restricted for controlled, and/or optimum, rate of conversion of substrate to target product.

Membrane unit 118a also prevents microorganisms of one vessel, for example bioreactor 102a, from substantially contaminating another vessel, for example bioreactor 102b. This microorganism isolation significantly reduces the chance of undesirable microorganisms being transferred from the first vessel to the second vessel. Isolation of bioreactor vessels also allows for greater control of the system 100 by allowing variables in isolated subsystems to be controlled to complement, or compensate for, the operation of other subsystems.

Although FIG. 2 shows the membrane unit 118a of FIG. 1, similar features may apply to membrane units 118b and 118n of FIG. 1. However, it is contemplated as a benefit of the invention, as explained further below, that differences in the membrane units may allow different bioreactors to operate under varying conditions.

Returning to FIG. 1, after passing through membrane unit 118a, the first permeate preferably does not include microorganisms to an extent significant enough to hinder the performance of the next bioreactor in series, bioreactor 102b. In one preferred embodiments, microorganisms in the permeate are 10⁻⁴ lower in concentration than in the retentate

The first retentate, which is returned to bioreactor 102a through processing line 120a, may include the target product, substrate which was not previously converted into the target product in bioreactor 102a, microorganisms including the fermentation microorganisms, and other non-substrate nutrients. In the event the fermentation microorganism cell mass in bioreactor 102a exceeds a desirable level, instead of returning all of the first retentate to bioreactor 102a, a portion of the first retentate, including the fermentation microorganism cell mass, may be bled off through processing line 124a. System 100 may include a microbe separation unit 126 for separating the microorganisms from the other components of the retentate. Microbe separation unit 126 may be any device for separating microorganisms from other components, such as but not limited to a membrane filter or a centrifugal separator. One example of a microorganism separator is disclosed in U.S. Pat. No. 4,460,687 issued to Ehnstrom, which is fully incorporated herein by reference.

Unneeded microorganisms may be sent to a microbe disposal unit 138 through processing line 128, and other components, such as the supernatant and/or clarified solution from microbe separator 126, may be conducted through processing line 130 and combined with the sterilized substrate in processing line 108 and reintroduced to bioreactor 102a. Although fermentation microorganisms may be recovered from the microbe separator 126 and reused in the system 100, in one preferred embodiment such reintroduction is avoided in order to minimize potential contamination. Fermentation microorganisms from the microbe disposal unit 138 may be sold as animal feed. Although not shown in FIG. 1, the other components from the microbe separator 126 may also be introduced to one of the other bioreactors of the system 100.

In operation, after bioreactor 102a receives a fermentation microorganism, such as yeast, and substrate, such as via processing line 108, the fermentation microorganism begins processing the substrate to yield the target product and byproducts such as carbon dioxide, low molecular weight organics, and some minerals and/or salts. The introduction of sterile oxygen or air through processing line 112a may be controlled to provide a greater amount of oxygen initially until the fermentation microorganism cell mass in bioreactor 102a reaches an optimal, or desired, level. Sterile oxygen or air may then be limited to a level sufficient to allow production of the target product, for example ethanol, while maintaining the desired fermentation microorganism cell mass, and preventing the overgrowth of cell mass that could undesirably limit production of the target product. In the case of ethanol, oxygen may be limited to allow anaerobic production of ethanol from yeast. Oxygen may be controlled between 0.05 and 0.55 cubic feet/cubic feet/minute of sterile oxygen or oxygen containing fluid.

A nitrogen-containing substance 114a may be introduced to bioreactor 102a as necessary for purposes including controlling the pH of the first mash and for providing when necessary for fermentation. Temperature in bioreactor 102a may also be controlled and monitored. In the case of ethanol production, the temperature is generally maintained at approximately 37° C. Agitation of the bioreactor 102a contents may be achieved through mechanical agitation devices known in the art, and/or by using the return from the membrane unit 118a.

Second bioreactor 102b may receive target product, sterilized substrate, sterilized oxygen, sterilized nutrients, fermentation microorganisms and substrate return. Processing line 122a may be used to introduce sterilized target product and substrate to second bioreactor 102b. Fermentation microorganisms may be input to bioreactor 102b before, during or after sterilized substrate may be introduced to bioreactor 102b. Oxygen, or gas or fluid containing oxygen, may be introduced to bioreactor 102b through oxygen sterilization unit 110 via processing line 112b.

Second mash may be continuously, or intermittently, taken from bioreactor 102b through processing line 116b and introduced to membrane unit 118b. Membrane unit 118b separates the contents of bioreactor 102b, the “second mash,” into a second retentate and a second permeate. The second retentate may be returned to bioreactor 102b through processing line 120b. The second permeate may be introduced to bioreactor 102n (where “n” indicates the number of bioreactors used in the system 100 is unlimited) through processing line 122b. After passing through membrane unit 118b, second permeate preferably does not include microorganisms to an extent significant enough to hinder the performance of the next bioreactor in series.

The second retentate, which is returned to bioreactor 102b through processing line 120b, may include the target product, microorganisms, substrate that was not converted into the target product in bioreactor 102a, and other non-substrate nutrients.

In the event the microorganism cell mass in bioreactor 102b exceeds a desirable level, instead of returning all of the second retentate to bioreactor 102b, a portion of the second retentate, including the microorganism cell mass, may be bled off through processing line 124b and introduced to the microbe separator 126.

In operation, after bioreactor 102b receives fermentation microorganisms and first permeate via processing line 122a, the fermentation microorganisms begin processing the first permeate to yield the target product and byproducts. The introduction of sterile oxygen or air through processing line 112b is restricted to control and/or optimize production while allowing fermentation microorganism cell mass maintenance as described above in regard to bioreactor 102a. A nitrogen-containing substance 114b may be introduced to bioreactor 102b as necessary for controlling the pH of the second mash and/or to add if required for microorganism fermentation activity. Second mash may be controlled to include a higher percentage of the target product, such as ethanol, than the concentration of the target product in the first mash.

The final bioreactor 102n may receive target product, sterilized substrate, sterilized oxygen, sterilized nutrients, fermentation microorganisms and substrate return. Processing line 122b, or a processing line associated with an intermediate bioreactor in the event the system 100 includes more than three bioreactors, may introduce sterilized target product and substrate to final bioreactor 102n. Yeast may be input to bioreactor 102n before, during or after sterilized substrate is introduced to bioreactor 102n. Oxygen, or gas or fluid containing oxygen, may be introduced through oxygen sterilization unit 110 through processing line 112n.

The final mash may be continuously or intermittently taken from bioreactor 102n through processing line 116n and introduced to the membrane unit 118n. Membrane unit 118n separates the final mash into a final retentate and a final permeate. The final retentate may be returned to bioreactor 102n through processing line 120n. The final permeate may be introduced to a final target product processor 132 through processing line 122n. After passing through membrane unit 118n, final permeate will preferably not include microorganisms to an extent significant enough to contaminate the target product.

Final retentate, which is returned to bioreactor 102n through processing line 120n, may include the target product, microorganisms, substrate which was not previously converted into the target product in bioreactor 102n, and other non-substrate nutrients.

In the event the microorganism cell mass in bioreactor 102n exceeds a desirable level, instead of returning all of the final retentate to bioreactor 102n, a portion of the final retentate, including the microorganism cell mass, may be bled off through processing line 124n and introduced to a microbe recovery unit 136. In alternative embodiments, the microbe recovery unit 136 may be omitted and a portion of the final retentate may be bled off and introduced to microbe separator 126. However, in the preferred embodiment, the final retentate is provided to a separate unit since it generally includes a lower percentage of lactose to recover and return to bioreactor 102a, and providing a separate microbe recovery unit 136 may decrease the chance of contamination.

In operation, after bioreactor 102n receives fermentation microorganisms and the second permeate, such as through processing line 122b, the fermentation microorganisms may begin processing the second permeate to yield the target product and byproducts. Again, the sterile oxygen or air through processing line 112n may be restricted to control and/or optimize production while allowing microorganism cell mass maintenance as described above in regard to bioreactor 102a. Again, a nitrogen-containing substance 114n may be introduced to bioreactor 102n as necessary for controlling the pH of the final mash.

Differing fermentation microorganisms may be used, and isolated, in the various bioreactors of system 100. Different combinations of fermentation microorganisms may be employed in the same bioreactor. The final mash may be controlled to include a higher percentage of ethanol than the contents of prior bioreactors in the system 100. In one embodiment, it is envisioned that the final mash may be controlled to operate at about 6.5% to 12% ethanol concentration; as a further example, if three bioreactors are used, second bioreactor 102b may operate at approximately 6-7% ethanol concentration, for example, while first bioreactor 102a may operate at approximately 3% ethanol concentration. Results will vary, of course, depending upon the variables described here including the robust nature of the microorganism, for example yeast, selected.

Target product concentrations in bioreactors 102a, 102b, and 102n may be varied in a number of ways, including but not limited to: varying the rate of introduction of substrate to the first bioreactor; varying cell mass in each bioreactor; varying the temperature in each bioreactor; and/or by diverting retentate to the microbe separator 126 and microbe recovery unit 136.

Final permeate may be introduced to the final target product processor 132. The target product may be stored in product storage unit 134. If microorganism cell mass concentration in bioreactor 102n exceeds an optimal or other setting, a portion of the final retentate may be diverted to a microbe recovery unit 136. The microbe recovery unit 136 may separate the microorganisms, for example yeast, and provide the microorganisms to the microbe disposal unit 138. The generally microorganism free portion of the microbe recovery unit 136 output may be provided to final target product processor 132.

Placing bioreactors in series, as in system 100, allows for optimal rates of substrate fermentation while permitting the minimization of total bioreactor size and the time required for production of the target product. The individual bioreactors may be configured to operate at different fermentation cell mass concentrations. Thus, some bioreactors may be permitted to operate with higher target product concentrations, while other bioreactors may be permitted to operate with higher cell mass concentrations. In system 100, the bioreactor 102n may be configured to operate at a low conversion rate associated with a high target product concentration, while bioreactor 102a operates at a high conversion rate with a lowered target product concentration.

Throughout the operation of system 100, fermentation variables such as temperature, pH, oxygen, nitrogen concentration, membrane process cross-flow rates, membrane permeation rates, turbidity, refractive indices, bioreactor agitation, and target product concentrations may be monitored and adjusted in each of the bioreactor vessels. Further, if desired, the fermentation variables may be independently adjusted in each of the bioreactor vessels.

Further, bioreactor vessels shown in FIG. 1 may be employed in series and in parallel to further enhance and increase production. For example, several bioreactors acting in parallel according to the description provided above in regard to bioreactor 102b may be employed to enhance and increase production.

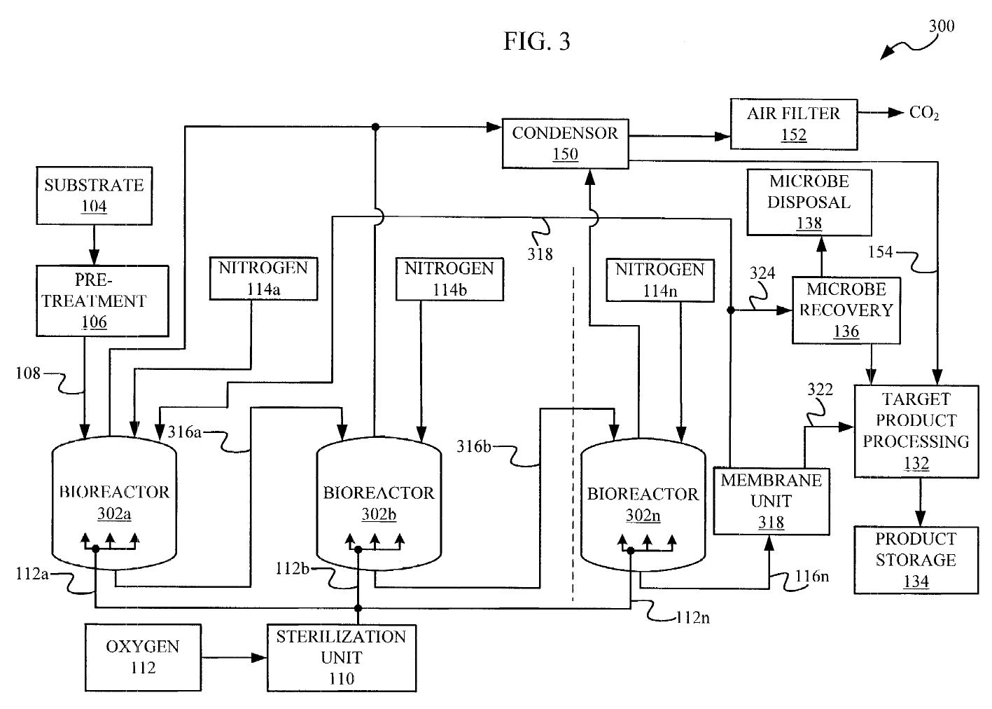

Now referring to FIG. 3, another embodiment of a bioreactor system, system 300, is shown in which incoming substrate may be at least partially processed into a target product, and fermentation microorganisms mass may be recovered and returned to the system 300. In particular, the fermentation microorganisms may be returned to the first of a plurality of bioreactor vessels. For example, fermentation microorganisms may be returned to bioreactor 302a shown in FIG. 3. Though three bioreactors are illustrated in FIG. 3, the actual number of bioreactors that may be incorporated into system 300 may be more or less and the number of vessels is unlimited. In FIG. 3, reference numbers for components that operate as described in regard to system 100 are retained. The substrate used in system 300 may be as described in regard to system 100.

First bioreactor 302a may receive sterilized substrate, sterilized oxygen, sterilized nutrients, fermentation microorganisms and substrate return. Fermentation microorganisms may be input to bioreactor 302a before, during or after sterilized substrate is introduced to bioreactor 302a.

In general during operation of the system 300, a portion of the content of a bioreactor vessel, the mash, may be continuously or intermittently removed from the bioreactor vessel and transferred to a succeeding bioreactor vessel. In the case of the last bioreactor 302n in series, the final mash may be transferred to final target product processor 132, or back to the initial bioreactor vessel 302a via a membrane unit 318.

A portion of the first mash may be continuously, or intermittently, removed from bioreactor 302a through processing line 316a and introduced to bioreactor 302b. A portion of the second mash may be continuously, or intermittently, removed from bioreactor 302b through processing line 316b and introduced to bioreactor 302n. Membrane unit 318 may separate the final mash into a first retentate, and a first permeate. First retentate may be returned to bioreactor 302a through processing line 318. First permeate may be introduced to final target product processor 132 through processing line 322.

Membrane unit 318 may be controlled to operate as desired for the desired operation of system 300. Membrane unit 318 may operate as described above in regard to membrane unit 118a of FIG. 1. However, membrane unit 318 may be designed for optimum, and/or desired, performance of the system 300. In operation, membrane 318 may provide a barrier through which microorganisms are restricted from passing from one portion of the membrane unit 318 to another in order that microorganisms may be restricted from final target product processor 132. Membrane unit 318 also may allow microorganism concentration to be increased and maintained at optimal levels, for example, 0.2 to 120 grams dry weight per liter in the bioreactors of system 300. For example membrane unit 318 may limit passage of microorganisms in order to maintain 0.2. to 120 grams per liter of yeast in bioreactor 302a. This permits microorganism concentrations to be limited for controlled, and/or optimum rate of conversion of substrate to target product in system 300.

In the event the microorganism cell mass in bioreactor 302a exceeds a desirable level, instead of returning all of the first retentate to bioreactor 302a, a portion of the first retentate, including the microorganism cell mass, may be bled off through processing line 324.

In operation, after bioreactor 302a receives fermentation microorganisms and substrate, such as via processing line 108, the fermentation microorganisms may begin processing the substrate to yield the target product and byproducts such as carbon dioxide. The sterile oxygen or air through processing line 112a may be controlled to provide a greater amount of oxygen initially until the microorganism cell mass in bioreactor 302a reaches an optimal level. Sterile oxygen or air may then be limited to a level sufficient to allow production of the target product, for example ethanol, while maintaining the desired microorganism cell mass and preventing the overgrowth of cell mass which could undesirably limit production of the target product. A nitrogen-containing substance 114a may be introduced to bioreactor 302a as necessary for controlling the pH of the first mash. In some applications, Nitrogen may be required in order for microorganisms to produce amino acids. While this may not be the case when dairy products are used as substrate, as they already contain some amino acids, it may be useful to add a basic solution, such as ammonia or ammonium salts, to raise the mash pH.) Temperature in bioreactor 302a may also be controlled and monitored.

Second bioreactor 302b may receive first mash through processing line 316a. Fermentation microorganisms may be input to bioreactor 302b before, during or after sterilized substrate is introduced to bioreactor 302b. Oxygen, or gas or fluid containing oxygen, may be introduced through oxygen sterilization unit 110 through processing line 112b. Second mash may be continuously, or intermittently, taken from bioreactor 302b through processing line 316b and introduced to bioreactor 302n.

In operation, after bioreactor 302b receives the first mash the fermentation microorganisms may begin further processing the first mash to yield the target product and byproducts. The sterile oxygen or air through processing line 112b may be restricted to control and/or optimize production while allowing cell mass maintenance as described above in regard to bioreactor 302a. A nitrogen-containing substance 114b may be introduced to bioreactor 302b as necessary for controlling the pH of the second mash and/or to add nitrogen for metabolism if necessary. Second mash may be controlled to include a higher percentage of ethanol than first mash.

The final bioreactor 302n may receive second mash from processing line 322b, or a processing line associated with an intermediate bioreactor in the event the system 300 includes more than three bioreactors. Fermentation microorganisms may be input to bioreactor 302n before, during or after sterilized substrate is introduced to bioreactor 302n. Oxygen, or gas or fluid containing oxygen, may be introduced through oxygen sterilization unit 110 through processing line 112n.

The final mash may be continuously, or intermittently, taken from bioreactor 302n through processing line 116n and introduced to the membrane unit 318. Membrane unit 318 separates the final mash into a final retentate and a final permeate. The final retentate may be returned to bioreactor 302a through processing line 318. The final permeate may be introduced to final target product processor 132 through processing line 322. After passing through membrane unit 318, final permeate preferably will not include microorganisms to an extent significant enough to contaminate the target product.

Final retentate, which may be returned to bioreactor 302a through processing line 318, may include the target product, microorganisms, substrate that was not converted into the target product in bioreactor 302n, and other non-substrate nutrients.

In operation, after bioreactor 302n receives yeast and second mash, such as via processing line 316b, the fermentation microorganisms may begin processing the second permeate to yield the target product and byproducts. The sterile oxygen or air through processing line 112n may be controlled to control and/or optimize production while allowing cell mass maintenance. A nitrogen-containing substance 114n may be introduced to bioreactor 302n as necessary for controlling pH of the last mash and/or to add nitrogen for metabolism if necessary. The final mash may be controlled to include a higher percentage of target product, such as ethanol, than the contents of prior bioreactors in the system 300. In one embodiment, the final mash may be controlled to operate at between about 6.5% to 12% ethanol concentration. Final permeate may be introduced to ethanol processor 132. The target product may be stored in product storage unit 134.

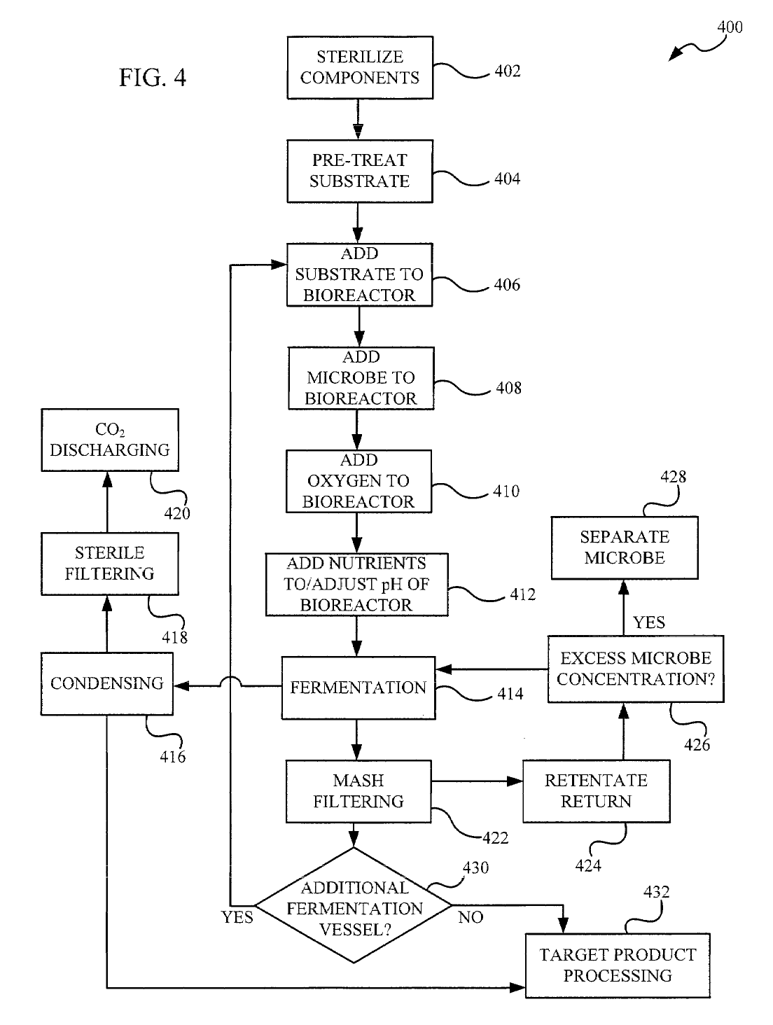

FIG. 4 is a flow chart illustrating a method 400 of processing of substrates, such as dairy products, into useful yields, such as ethanol. Though methods of practicing the invention are not limited to the system components shown in FIGS. 1 to 3, as non-limiting examples the components of FIGS. 1 to 3 may be referenced in describing the steps of practicing the embodiments illustrated in FIG. 4.

In step 402, fermentation system components may be sterilized. The components may include bioreactor vessels and processing lines, such as those shown in FIGS. 1 and 2. The components may be sterilized using a variety of methods, including steam and chemical sterilization. Chemical sterilization may include chlorine and caustic cleaning substances.

In step 404, the substrate may be pre-treated. Step 404 may include passing the substrate through a pre-treatment unit at a concentration of 4-25% solids by weight. Pre-treatment may include, but is not limited to, thermal pasteurization, thermal sterilization, chemical sterilization, UV sterilization, and filtration sterilization. Sterilization filtration may use a variety of filters, including a 0.02 micron membrane filter.

In step 406, the sterilized substrate may be introduced to a bioreactor. In step 408, a fermentation microorganism may be introduced to a bioreactor. The fermentation microorganism may be introduced before, during or after the substrate is introduced to the bioreactor. In step 410, sterile oxygen, or fluid containing oxygen, may be introduced to the bioreactor. The oxygen may be controlled to provide a large quantity initially to promote microorganism cell mass growth until optimum growth is achieved, and then limited to optimize production of a target product while allowing sufficient cell mass growth to maintain stability. In step 412, nutrients may be introduced, and pH adjustments may be made, to the bioreactor. In step 414, a mash in the bioreactor may be allowed to ferment to produce the target product.

Step 414 may produce gases such as carbon dioxide (CO₂). In step 416, vapors that may include carbon dioxide and target product may pass through a condenser. In step 418, the vapors may be passed through a sterilizing vent filter. In step 420, carbon dioxide may be discharged.

In step 422, a portion of the mash may be filtered to separate a retentate from a permeate. Step 422 may include introducing a portion of the mash to a membrane unit. Step 422 may include the step of cleaning a filtration device.

In step 424, all or a portion of a first retentate may be returned to step 414 where it is subject to further fermentation. In step 426, in the event the microorganism cell mass in the bioreactor exceeds a desired level, all or a portion of the retentate may be provided to a microbe separator.

In step 428, the retentate may be separated into a microorganism containing solution and a clarified solution. In step 430, if a further bioreactor fermentation process is desired, steps 406 through 428 may be repeated using an intermediate bioreactor fermentation vessel. In step 430, if a further bioreactor fermentation process is not desired, the target product may be separated from the permeate in step 432. In step 432, any target product recovered in step 416 may also be separated from other materials.

Referring to FIGS. 1-4, it should be understood that carbon dioxide and target product vapors may pass through a condenser 150 and a sterile filter 152 to permit sterile bleed from the bioreactors and recovery of some of the target product vapors, to reduce discharge pollutants while bolstering target product yield. Target product may be passed from the condenser 150 to the target product separator on processing line 154. The sterile filter may be used maintain the sterility of the system and to prevent contaminating microorganisms from entering the system.

In general, the use of multiple reactors in series and membrane technology provides that as the substrate is moved from one reactor to the next, the target product (e.g., ethanol) concentration increases, while the substrate concentration (e.g., lactose) decreases. In other words, in a continuous fermentation process of the present invention, multiple bioreactors/fermentors are arranged so that permeate from the first bioreactor in series may be fed to the second bioreactor in series, and so on, until permeate from the last bioreactor in series constitutes the feed to the distillation train.

It will be understood that the number of bioreactors that may be used in the fermentation train ranges from a minimum of one bioreactor to as many bioreactors in series and parallel as desired and as economically tenable. Parallel bioreactors, for example, may operate together as one unit or as part of a separate, but concurrent, fermentation train.

It should also be recognized that in the continuous fermentation processes disclosed here, the biomass may consist of a single strain of a microorganism (e.g., yeast), several strains, or a combination of two or more different microorganisms, working together to ferment the substrate. In the case of multiple organisms, they may be combined together in each bioreactor, or they may be segregated by the membranes to act singly in any bioreactor in the fermentation train.

It should also be understood that the size of the fermentation train may be significantly reduced by separating the microorganisms using (e.g.) cross-flow (tangential flow) membrane filtration technology, and then returning the microbial biomass back to the fermenter. This may be accomplished by building and maintaining a relatively high level of biomass concentration in each fermenter through the methods disclosed above. A small amount of fermentation broth may, or may not be, bled off each bioreactor to maintain an optimal microbial cell dry weight concentration between (e.g.) 0.2 g/l and 100 g/l. It will be understood that a small fermentation train is easier to control, easier to automate, requires smaller bioreactor vessels, and has a smaller footprint, resulting in reduced up-front capital costs for the entire system.

It should also be understood that each bioreactor may be provided with its own cross-flow membrane filtration system to retain the cell mass and direct it back to the desired bioreactor. Alternatively, any combination of bioreactors may have a membrane system associated with it, for recovery and recycle of the cell mass. Permeate may be allowed to pass through the membrane on to the next bioreactor in the fermentation train. Permeate may contain the fermentation product, any unutilized feed substrate, and any unutilized non-substrate nutrient agents but without the cell mass. As noted above, the membranes used may be hollow fiber, flat sheet plate and frame, flat sheet spiral, tubular configurations, or any combination of these configurations as may be economically feasible. The membrane may be constructed from organic materials or inorganic materials. The membranes may have a pore size that can vary from 150 angstroms in diameter to 1.0 microns in diameter, for example. Operating pressures for the membrane system may vary from 5 psig to 150 psig, for example.

In an alternative system design, shown in FIG. 3, a membrane barrier may be used on the last bioreactor/fermenter only, and the microbial cells may be returned back to the first fermenter or to any other fermenter in the fermentation train.

As desired, the biomass bled off of each bioreactor to maintain desired cell concentrations may be run through a separate membrane unit or a high speed centrifuge to separate out the biomass for disposal. Permeate or supernatant, respectively containing residual amounts of substrate may be directed back to the bioreactor. This can improve yield while allowing enhanced control over the biomass concentration.

The bioreactors may be agitated with a mechanical agitation device or agitated using the return flow from the membrane system.

The cross-flow membranes will preferably retain most of the microorganisms in each bioreactor, effectively performing a non-heat pasteurization of the substrate as it moves through the fermentation train from bioreactor to bioreactor. This will help minimize and isolate infection of the bioreactors during the continuous fermentation process.

Chemical stabilization of the fermentation process may be achieved by introducing hydrogen peroxide or sodium sulfite or both to the feed substrate at the whey permeate production site, or by adding the same to a contaminated bioreactor to exclude non-yeast microorganisms. Preferably, the chemical stabilization will have a minimum effect upon desired fermentation microorganisms. Effective concentrations of hydrogen peroxide and sulfite are between 10 ppm and 500 ppm each, for example.

Sterile pure oxygen or sterile oxygen containing air may be carefully introduced into each bioreactor and controlled continuously to allow the microorganisms to remain viable. The amount of oxygen introduced should be controlled as too much oxygen may allow biomass over-production, reducing ethanol yield while increasing cell mass disposal; on the other hand, the presence of too little oxygen may cause the microorganisms to die off, reducing the efficiency of the bioreactor and causing the fermentation train to operate sub-optimally or not at all for the desired long-term continuous runs. As one preferred exemplary range, the levels of oxygen may be controlled between 0.05 and 0.55 ft³/ft³/min of sterile oxygen or air per bioreactor.

It will be understood that the feed to the fermentation process should be properly handled by pasteurizing/sterilizing it after it is produced, and the substrate should be transported either hot or cold and preferably in concentrated form. The substrate should be free of other microbial growth which, if present, may introduce metabolic by-products that may inhibit the fermentation process. Clean feed stock can be vital to proper operation of the fermentation process.

To further bolster target product production, reactor vapors may be directed through a condenser (see condensing step 416, FIG. 4). Condensed target product may be directed for further processing (see step 432, FIG. 4), remaining vapors may then be processed in a sterile filtering step 418, followed by CO₂ discharging step 420. The condensed, target product may then be directed to a target product separation step 132 (see FIG. 3) (e.g., the still for ethanol). Alternatively, the condensate may be directed to the final bioreactor vessel, for example.

As an alternative to or in addition to a membrane filter(s), a centrifugal separator(s) or other separating or filtering mechanism (“microorganism refiner” as termed in the claims) may be used, particularly in the last bioreactor vessel.

It should now be apparent to persons of ordinary skill in the art that by employing membrane-enhanced, continuous fermentation systems, higher yields of fermentation target product (e.g., ethanol) may be realized; at the same time, manpower costs may be lowered by using process automation. Overall, a more economical fermentation process is believed to be provided.

The above description is not intended to limit the meaning of the words used in the following claims that define the invention. For example, while several possible designs have been described above, persons of ordinary skill in the art will understand that a variety of other designs still falling within the scope of the following claims may be envisioned and used. It is contemplated that these or other future modifications in structure, function or result will exist that are not substantial changes and that all such insubstantial changes in what is claimed are intended to be covered by the claims.

I claim:

1. A continuous-flow fermentation system for providing a target product from a substrate, comprising:
at least first and second reaction vessels in selective fluid communication, the first reaction vessel being charged with a first mash that includes the substrate and reactive microorganisms, the reactive microorganisms comprising fermentation microorganisms for use in converting the substrate into the target product;
the second reaction vessel being charged with a second mash that includes the substrate and reactive microorganisms, the second reaction vessel receiving a selective portion of the first mash from the first reaction vessel;
a membrane filter located between the first and second reaction vessels and configured to separate the first mash from the first reaction vessel into at least a first retentate and a first permeate; and
a microorganism refiner located downstream from the second reaction vessel;
wherein the reactive microorganisms within the first reaction vessel are generally retained in the first retentate by the membrane filter and returned to the first reaction vessel, and the first permeate which is substantially free of microorganisms is introduced to the second reaction vessel, and wherein the reactive microorganisms within the second reaction vessel form a second retentate which is generally retained in the second reaction vessel by the microorganism refiner;
whereby each separate reaction vessel is capable of substantially retaining the reactive microorganisms initially resident in that separate vessel.

2. The system of claim 1, wherein a portion of either the first and/or the second retentate is removed from its corresponding reaction vessel, reactive microorganisms are removed this portion, and the remainder of the retentate is transferred back to at least one of the reaction vessels.

3. The system of claim 1, wherein the microorganism refiner comprises a centrifugal separator.

4. The system of claim 1, wherein the microorganism refiner comprises a membrane filter.

5. The system of claim 1, wherein the reactive microorganisms charging the first and second reaction vessels comprise the same type of reactive microorganisms.

6. The system of claim 1, wherein the reactive microorganisms charging the first and second reaction vessels comprise different types of reactive microorganisms.

7. The system of claim 1, wherein the reactive microorganisms charge the second reaction vessel at a time subsequent to the charging of the first reaction vessel with the reactive microorganisms.

8. The system of claim 1, wherein the second reaction vessel contains a second mash, and wherein the second mash has a different percentage of the target product than the first mash.

9. The system of claim 8, wherein the second mash has a higher percentage of the target product than the first mash.

10. The system of claim 1, wherein the second reaction vessel contains a second mash, and wherein the second mash has a different substrate concentration than the first mash.

11. The system of claim 10, wherein the second mash has a lower substrate concentration than the first mash.

12. The system of claim 1, wherein the second reaction vessel contains a second mash, and wherein the second mash has a different microorganism concentration than the first mash.

13. The system of claim 12, wherein the second mash has a lower microorganism concentration than the first mash.

14. The system of claim 1, further comprising a third reaction vessel designed to ferment substrate and reactive microorganisms and located downstream from and in selective fluid communication with the second reaction vessel, and a microorganism refiner located downstream of the third reaction vessel.

15. The system of claim 1, further comprising a third reaction vessel designed to ferment substrate and reactive microorganisms and processing in parallel with the first reaction vessel.

16. The system of claim 1, wherein the reactive microorganisms also comprise contaminating microorganisms.

17. The system of claim 16, wherein the contaminating microorganisms are isolated in the first reaction vessel by the membrane filter.

18. The system of claim 1, wherein the fermentation microorganisms comprise different types of fermentation microorganisms, each for use in converting the substrate into the target product.

19. The system of claim 18, wherein the fermentation microorganisms comprise at least first and second fermentation types of microorganisms, and wherein the first type of fermentation microorganism is capable of more efficiently processing the substrate into the target product in the presence of relatively greater concentrations of alcohol than the second type of fermentation microorganism.

20. The system of claim 1, wherein the second mash includes a type of fermentation microorganisms different from the fermentation microorganisms present in the first mash.

21. The system of claim 20, wherein the first type of fermentation microorganism comprises Saccharomyces fragilis and the second type of fermentation microorganism comprises Saccharomyces cerevisiae.

22. The system of claim 1, wherein the first reaction vessel is hermetically sealed.

23. The system of claim 1, wherein sterilized oxygen is used to grow the fermentation microorganism.

24. The system of claim 1, wherein a nitrogen-containing substance is used to control the pH of the mash in each bioreactor.

25. The system of claim 1, wherein the nitrogen-containing substance comprises ammonia or ammonium salts.

26. The system of claim 1, wherein the substrate comprises one or more of the following: lactose; whey; whey permeate; corn; wheat; rye; rice; potatoes; artichokes; sugar beets; sugarcane; fruits; plant fiber; wood by-products; paper; and/or grasses.

27. The system of claim 1, wherein the substrate comprises lactose concentrated using a reverse osmosis system.

28. The system of claim 1, wherein the target product comprises one or more of the following: ethanol; propanol; isopropanol; butanol; and/or acetone.

29. The system of claim 1, wherein the membrane filter comprises one or more of the following materials or classes of materials or substances: cellulosic; polyvinylidene fluoride; polyether sulfone; polysulfone; ceramic; sintered stainless steel; and/or graphite.

30. The system of claim 1, wherein the membrane filter is configured in a shape comprising one or more of the following shapes: hollow fiber; flat sheet spiral; flat sheet plate; frame and/or tubular.

31. The system of claim 1, wherein the membrane filter characteristics are capable of being altered during the operation of the system by an electrical charge directed on or through the membrane.

32. The system of claim 1, wherein the membrane filter has pores that have a diameter between 150 angstroms and 1 micron.

33. The system of claim 1, wherein the membrane filter is configured to operate at pressures between 5 to 150 psig.

34. The system of claim 1, wherein the target product is produced in an anaerobic reaction.

35. The system of claim 1, wherein the target product is produced in an aerobic reaction.

36. The system of claim 1, wherein the substrate is treated to exclude contaminating organisms.

37. The system of claim 1, wherein the membrane filter has a isotropic morphology.

38. The system of claim 1, wherein the membrane filter has a anisotropic morphology.

39. A method of continuously fermenting a mash including a substrate and reactive microorganisms to convert the substrate into a useful target product, comprising the steps of:
providing at least first and second reaction vessels in selective fluid communication in a continuous flow system, the first reaction vessel containing a first mash that includes a substrate and reactive microorganisms useful in converting the substrate into a target product, and the second reaction vessel containing a second mash that includes the substrate and reactive microorganisms; and
locating a membrane filter downstream from the first reaction vessel and configured to receive at least a portion of the first mash and to separate the first mash into at least a first retentate and a first permeate, wherein the reactive microorganisms are generally retained in the retentate by the membrane filter and returned to the first reaction vessel;
locating a microorganism refiner downstream from the second reaction vessel and configured to receive at least a portion of the second mash and to separate the second mash into a second retentate and a second permeate, wherein the reactive microorganisms are generally retained in the second retentate by the microorganism refiner and returned to the second reaction vessel; and
introducing the first permeate which is substantially free of microorganisms to the second reaction vessel;
whereby each separate reaction vessel substantially retains the microorganisms initially resident in that separate vessel.

40. The method of claim 39, further comprising the step of recovering the target product from vapors emanating from the reaction vessels.

41. The method of claim 40, wherein the recovering step includes directing the vapors through a condenser, and then directing the condensed target product through a target product separation processor.

42. The method of claim 39, further including the step of condensing vapors and the step of passing vapors through a sterilizing filter.

43. The method of claim 39, wherein the substrate is chemically stabilized.
